(19) United States
(12) Patent Application Publication
(10) Pub. No.: US 2025/0339477 A1
(43) Pub. Date: Nov. 6, 2025

(54) TREATMENT OF THERAPY-INDUCED ENTEROPATHY

(71) Applicant: ILYA PHARMA AB, Uppsala (SE)

(72) Inventors: Evelina VÅGESJÖ, Uppsala (SE); Emelie ÖHNSTEDT, Uppsala (SE); Cristian DONAS CUADRA, Uppsala (SE); Mia PHILLIPSON, Uppsala (SE); Stefan ROOS, Uppsala (SE)

(21) Appl. No.: 18/725,695

(22) PCT Filed: Dec. 28, 2022
(86) PCT No.: PCT/EP2022/087964

§ 371 Date: Jun. 28, 2024

(30) Foreign Application Priority Data
Dec. 29, 2021 (GB) 2119079.8
Mar. 28, 2022 (GB) 2204387.1

(51) Int. Cl. A61K 35/744; A61K 35/00; A61K 38/19; A61P 1/04

(52) U.S. Cl. CPC A61K 35/744; A61K 38/195; A61P 1/04; A61K 2035/115

(57) ABSTRACT

The present invention provides lactic acid bacteria (LAB) which have been engineered to express a therapeutic protein capable of promoting resolution of inflammation and/or wound healing for use in treating or preventing therapy-induced enteropathy in a human or animal subject.

### FIELD

The invention relates generally to the treatment of enteropathy which is induced in a subject as a result of therapy administered to the subject, for example in the treatment of cancer. In particular, the treatment comprises the administration of lactic acid bacteria which have been engineered recombinantly to express a therapeutic protein capable of promoting resolution of inflammation and/or wound healing.

### BACKGROUND

Various therapies administered to subjects for the treatment or management of their clinical conditions, including notably cancer, may have damaging or untoward side effects, including particularly in the lower gastrointestinal (GI) tract. Such therapy-induced enteropathy may limit the application of the therapy in question, and more particularly may be a cause of significant patient morbidity.

The introduction of immune checkpoint inhibitors (ICIs) targeting, for example, cytotoxic T lymphocyte-associated protein 4 (CTLA-4), programmed cell death receptor 1 (PD-1), and programmed death ligand 1 (PD-L1) has improved the prognosis of many advanced cancers, including melanoma, urothelial and renal cell carcinoma, and non-small cell lung cancer. Although treatment with ICIs may facilitate effective tumor control in the responders, many patients treated with ICIs will develop immune-mediated adverse events such as enterocolitis, hepatitis, arthritis, dermatitis, thyroiditis, and hypophysitis.

Furthermore, although combination ICIs are more effective for cancer control compared to monotherapy, this strategy is associated with a higher risk of immune-mediated adverse events, dependent on dose and duration of treatment. Immune-mediated enterocolitis, characterized by abdominal pain and diarrhea, will develop in up to one-third of patients treated with ICIs, which negatively affects patient quality of life and potentially limits the persistence of ICI therapy. However, progression-free survival is also highest among patients who develop immune-related gastrointestinal AEs and is a proxy for, for example, enhanced T lymphocyte activity with immunologic tumor suppression. Therefore, effective prevention or treatment or of ICI-induced enterocolitis is intrinsically an important component of the long-term oncologic management plan.

Current guidelines recommend treating ICI-induced enterocolitis based on the grade of presentation as evaluated using the Common Terminology Criteria for Adverse Events (CTCAE). Supportive measures, systemic corticosteroids, temporary or permanent discontinuation of ICI treatment, and the TNF inhibitor infliximab are sequentially recommended and reported for progressively more severe disease. However, the potential counterproductive impact of TNF inhibitors on the effect of the ICI is a concern and has been reported. There are several limitations with this current paradigm. First, CTCAE grading is based on clinical symptoms including stool frequency, bleeding, abdominal pain, and fever, rather than more objective markers of the activity of the disease. Second, there are no validated instruments to date to describe and measure endoscopic and histological disease activity for treatment induced enteropathy and occasionally biopsies are taken for diagnosis and control as the clinical symptoms may not fully capture the severity of the colitis for satisfactory assessment of risk of ruptures. Third, there is limited evidence to inform therapeutic decisions, either for prevention or treatment of ICI-induced enterocolitis. Clinically relevant questions regarding the optimal timing, sequence, and duration of treatment remain unanswered. Finally, it remains unclear what the role of established systemic treatments for inflammatory bowel disease (IBD, including ulcerative colitis and Crohn's disease) aside from infliximab play in the management of ICI-induced enterocolitis.

In this regard, whilst ICI-induced enterocolitis does share certain histological features with acute colitis such as is observed with IBD, the two entities definitely appear to be immunologically and histopathologically distinct from each other. In particular, we have observed an increase in fibrosis and epithelial ulcers in the colon associated with ICI-induced colitis, not necessarily correlating with the traditional view going hand in hand with the traditional clinical signs associated with colitis. It has also been reported that pathogenesis in ICI-induced colitis is predominantly driven by T-cells, whereas humoral (B-cell) immunity has been shown to be more important in IBD (see for example Yanai et al., Clin. Gastroenterol. Hepatol. 2017, 15: e8081; and Bertha et al., ACG Case Rep. J. 2017; 4^{th} 112). Furthermore, the different forms of colitis in their acute form appear to be clinically distinct from one another, for example in terms of severity and potential for rapid progression of complications. In addition, it is not yet clear whether the chronic colitis that has been observed in patients after the ICI therapy is completed is similar to long-term IBD colitis disease (Hsieh et al., BMJ Case Rep. 2016, bcr-2016-216641).

Another form of commonly-observed therapy-induced enteropathy is radiation-induced enteropathy. Intestinal radiation toxicity (radiation enteropathy) is generally classified as early (acute) when it occurs within 3 months of radiation therapy or delayed (chronic) when it occurs more than 3 months after radiation therapy. Whilst the incidence of severe (grades 3-4) delayed intestinal radiation toxicity has diminished over time, largely thanks to improvements in radiotherapy planning and radiation delivery techniques, it has been reported that roughly half of radiotherapy patients will have some form of chronic GI dysfunction. Delayed radiation enteropathy is a chronic often progressive disorder, and is associated with substantial long-term morbidity.

As noted above, the occurrence of ICI-induced enterocolitis appears to be associated with a better oncological response to the ICI treatment. Recent data indicates that remission in ICI-induced enterocolitis after treatment with the TNF inhibitor infliximab may be associated with less response to the immune checkpoint inhibitor, indicated by more cancer progression. Based on this, we speculated that local immunomodulatory therapy in the inflamed colon, without systemic global effects of dampening the immune system, that could negatively impact the anti-cancer effect of the ICI, could provide the right balance between immunosuppression and immune activation. This would represent an attractive approach to treat the growing unmet medical need for further or improved treatments for ICI-induced enteropathy, and indeed for radiation-induced or other therapy-induced enteropathies. In WO 2016/102660 we describe the use of lactic acid bacteria genetically

modified to express various proteins with wound healing activity, notably CXCL12, CXCL17 and Ym1, to promote the healing of wounds. The effects attributed to the bacterially-expressed proteins reported in this document are believed to arise from their immunomodulatory effects on local immune cells in the vicinity of the wound. We have now expanded this work into the area of therapy-induced enteropathies.

### SUMMARY

We propose that lactic acid bacteria (LAB) which are modified, according to the disclosure herein, to express certain proteins which are useful for promoting resolution of inflammation and/or wound healing, may be used in the specific context of treating therapy-induced enterophathies.

The differences noted above between ICI-induced and IBD-associated enterocolitis and the accentuated need of local immune suppression in ICI induced enterocolitis suggest that IBD therapies may not be effective or suitable in the ICI-induced context. Despite this, we nonetheless believed that the effects of resolution of inflammation and wound healing proteins expressed by LAB on local B-cells, T-follicular helper cells, macrophages and other immune cells in the local immune cell rich areas and area of damage induced in the gut by ICI or other therapy would be of benefit in ameliorating and treating this damage. We have tested this proposal and have shown beneficial effects of administering the modified LAB to animal cancer models with colitis and or treated with ICI therapy in reducing disease activity, crypt damage and epithelial loss (erosion and ulceration of crypt epithelium) in the colon assessed blinded using conventional histopathology. Further, an effect of the modified LAB has also been shown in reducing colon-shortening induced by ICI therapy. Surprisingly, the beneficial effects of the treatment with the modified LAB were observed on manifest disease during the ICI treatment, and thus this is not just an effect of impeding the development of disease. Rather it has been shown that manifest signs of damage in the GI tract can be treated, supporting that the LAB may be administered after damage induced by the therapy has been incurred. Importantly, the beneficial effects of the modified LAB were not observed with the corresponding unmodified LAB (i.e. the wild-type bacteria, which have not been modified to express the wound healing protein).

Accordingly, in a broad and first aspect, we provide herein engineered lactic acid bacteria (LAB) for use in treating or preventing therapy-induced enteropathy in a subject, wherein said bacteria have been engineered to express a protein which promotes resolution of inflammation and/or wound healing.

In particular, the engineered LAB are for use in treating therapy-induced enteropathy.

In a particular embodiment the therapy-induced enteropathy is oncotherapy-

induced enteropathy, and more particularly enteropathy induced by ICI therapy or radiation therapy.

The subject is a human or animal subject.

In particular, the protein is a mammalian protein, and more particularly it is an immunomodulatory protein. In an embodiment, the protein modulates the growth and/or activity of immune cells, and particularly macrophage cells or their precursors. In an embodiment, the said protein is a cytokine or chemokine. In one embodiment, the protein is a CXC protein.

In a more particular embodiment, the protein is selected from CXCL12, CXCL17 and Ym1, particularly CXCL12 or CXCL17. In another embodiment the protein is TGF-β.

The LAB may be any genus, species or strain of LAB, but as discussed further below, the LAB are particularly Lactobacilli. In an embodiment the LAB are Limosilactobacillus reuteri, formerly known as Lactobacillus reuteri.

In another but related aspect, provided herein is a pharmaceutical composition comprising herein engineered lactic acid bacteria (LAB) for use in treating or preventing therapy-induced enteropathy in a subject, wherein said bacteria have been engineered to express a protein which promotes resolution of inflammation and/or wound healing.

Still another aspect provides use of engineered lactic acid bacteria (LAB) for the manufacture of a medicament for use in treating or preventing therapy-induced enteropathy in a subject, wherein said bacteria have been engineered to express a protein which promotes resolution of inflammation and/or wound healing.

Yet another aspect provides a method for treating or preventing therapy-induced enteropathy in a subject, said method comprising administering to a subject who has been or is being administered an enteropathy-inducing therapy, engineered lactic acid bacteria (LAB) which have been engineered to express a protein which promotes wound healing.

### DETAILED DESCRIPTION

The medical uses and methods herein are directed to the treatment or prevention of therapy-induced enteropathy, particularly the treatment thereof.

The therapy which induces the enteropathy is not limited and may be any therapy which when administered to a human or animal subject causes or results in enteropathy in the subject. Particularly, however, the therapy is a therapy for cancer, or in other words an oncotherapy. Accordingly, in an embodiment the subject is suffering from or has been diagnosed with cancer (i.e. is a cancer patient).

The cancer is not limited and can be any cancer. In particular, the cancer may be a cancer which is suitable for or susceptible to treatment with an immune checkpoint inhibitor (ICI). The cancer may for example be melanoma, urothelial or renal cell carcinoma, or lung cancer, e.g. non-small cell lung cancer, but these are merely representative examples. The cancer may be characterized by solid tumors. In an embodiment, the cancer may be an advanced cancer. The nature of the cancer is not critical to the proposed uses. The cancer may accordingly be a cancer of any organ or tissue in the body. However, in one embodiment, the cancer does not include (or the cancer is not) colorectal cancer.

The enteropathy-inducing therapy may be a therapy with any therapeutic agent, including a pharmacological or pharmaceutical agent, including an immunotherapy (e.g. an immunotherapeutic agent), or radiotherapy. The therapy may thus involve the administration to the subject of radiation or of a small molecule pharmaceutical, e.g. a chemotherapeutic agent, or a biological molecule, e.g. a protein, for example an antibody, or an antibody-derived or antibody-based protein.

In an embodiment the therapy-induced enteropathy is immune checkpoint inhibitor (ICI)-induced enteropathy, or radiation-induced enteropathy (which term is synonymous with “radiation enteropathy”). In a more particular embodiment, the radiation-induced enteropathy is delayed radiation-induced enteropathy.

The term “enteropathy” is used broadly herein to include any damage, injury or inflammation to the gut, or in other words the GI tract, particularly, in the lower GI tract, notably the small and large intestines. It may include any histological changes to the GI tract, as compared to before the treatment or to a healthy individual who has not received the enteropathy-inducing therapy. More particularly, these may be histopathological changes.

The histological features of therapy-induced enteropathy include cryptitis, intra-epithelial neutrophilic lymphocytes, glandular destruction, erosions of the mucosal surface, e.g. mucosal ulcerations, crypt abscesses, apoptosis and necrosis. Other symptoms include diffuse erythema, oedema, loss of vascularity, increasing to haemodynamic instability, serious congestion, ischaemic bowel, and perforations. Generally speaking, inflammatory changes may be seen at foci in the intestines and the foci may have different size. Accordingly, the enteropathy to be treated herein may be defined as inflammation or inflammatory changes in the GI tract, or more particularly in the intestines. The inflammatory changes may precede, and may be seen before or after, overt inflammation is detected. Such changes may lead to shortening of the colon or increase in colon weight. As noted above, the severity of the therapy-induced enteropathy may be graded using the Common Terminology Criteria for Adverse Events (CTCAE) (grades 1-4, increasing in severity). Generally speaking, the enteropathy may be manifest as colitis, that is inflammation of the colon. However, the term enteropathy includes lower grades of damage or inflammatory changes, including those not yet manifest as overt colitis by traditional clinical assessments. In one particular embodiment, the therapy-induced enteropathy to be treated herein may be characterized by crypt damage, epithelial loss and/or colon shortening or weight increase.

Further, in some cases the therapy-induced enteropathy may alternatively or additionally be characterized by fibrosis in the intestines. As noted above, we have recently observed that fibrosis and oedema may be seen in cases of ICI-induced enteropathy in mouse models of the disease, where the more traditional clinical signs of colitis, or overt colitis, are not measurable, or are not yet seen. The observations of colon shortening seen in these studies of ICI-induced colitis of >20% reduction are at par with the most severe grades of colitis in other models, e.g. chemically induced colitis using dextran sulfate sodium (DSS) that presents mainly with clinical signs as measured by weight loss, diarrhea and blood in stools.

Commonly-used markers to determine or assess disease activity in inflammatory bowel disease (IBD) or colitis may be used to assess therapy-induced enteropathy. The therapy-induced enteropathy may thus also be detected and/or diagnosed, at least in part, by detecting inflammatory markers, including for example, pro-inflammatory cytokines, calprotectin, and/or the presence or activity of immune cells, including e.g. T-cells in the gut.

The clinical signs of enteropathy include abdominal pain, increased numbers of bowel movements per day, diarrhea, rectal bleeding and mucus in the stools. The clinical symptoms may interfere with active daily living. With increasing severity, at stage 4, symptoms may include haemodynamic instability, serious congestion, ischaemic bowel, perforations, megacolon, and sepsis. Ultimately this could lead to death.

Therapy-induced enteropathy may be diagnosed based on one or more clinical symptoms, including notably diarrhea, and measurement of serum inflammatory markers and electrolytes. Significant abnormalities indicative of therapy-induced enteropathy include anaemia, increased C-reactive protein, and slow serum albumin levels. The diagnosis may be confirmed by endoscopic or rectoscopic examination, and/or by biopsy. Interestingly, murine models have demonstrated that ICI-induced enteropathy does not necessarily display the common clinical features seen in other types of enteropathy and IBD such as weight loss, diarrhea and blood in stool, and instead exhibits clinical features such as increased fibrosis development and epithelial ulcers, particularly within the colon (Examples 3 and 4). Thus, increased fibrosis may also be assessed as a clinical marker of therapy-induced enteropathy. Fibrosis may result in colon-shortening, which may be observed as an indicator of fibrosis in the colon.

Common clinical features of radiation-induced enteropathy include altered intestinal transit, malabsorption, and dysmobility, Severe cases may progress to intestinal obstruction, fistula formation or frank intestinal perforation. Accordingly, the medical uses herein include the treatment of overt or manifest clinical disease, or pre-clinical conditions.

That is, the enteropathy to be treated includes manifest or overt disease, as demonstrated by clinical symptoms, e.g. diarrhea or symptoms of colitis, as well as pre-clinical enteropathy, where clinical symptoms are not yet seen, but damage to the gut has occurred, as determined by histological features, or changes, e.g. as discussed above, including particularly fibrosis and epithelial ulcers, especially in the colon. Thus, the enteropathy may be diagnosed by overt symptoms, and/or by examination of the GI tract (e.g. by histological examination, e.g. by biopsy, or by endoscopic examination).

The medical uses herein include treatment of established disease, e.g. where the engineered LAB are administered after therapy-induced enteropathy has been identified or diagnosed in a subject, and this represents a particularly advantageous aspect of the therapies disclosed herein. In particular, the therapies herein involve the treatment of therapy-induced colitis once colitis is manifest. However, the LAB may also be used in prevention, that is to delay or prevent the development of therapy-induced enteropathy. In such a case, the LAB may be administered to the subject at the time of the start of treatment, before the enteropathy has developed, or before it has been diagnosed, e.g. before there is overt or manifest colitis. For example, the LAB may be administered together with the therapy, or shortly after. In such a case, the LAB may be used protect against the development of colitis.

In an embodiment, the engineered LAB may be used in the treatment of subjects undergoing therapy who exhibit increased fibrosis and/or epithelial ulcers, or risk thereof, in the intestines, particularly in the colon (i.e. as a result of said therapy).

In another embodiment, the engineered LAB may be used to inhibit the development of fibrosis and/or epithelial ulcers in the intestines, particularly the colon, of a subject undergoing therapy. The therapy may be any therapy as discussed herein, which may be referred to as an enteropathy-inducing therapy. The term “inhibit” as used herein includes any effect in limiting the fibrosis and/or epithelial ulcers, e.g. reducing, preventing, and/or delaying fibrosis and/or epithelial ulcers. The subject may accordingly be a subject at risk of developing therapy-induced enteropathy.

In another embodiment, the subject presents, or is classified as, greater or equal to stage 2 of therapy-induced colitis according to the CTCAE.

In another embodiment, the engineered LAB may be used to revert to or maintain the severity of the therapy-induced colitis at less than or equal to grade 2 according to CTCAE.

In an embodiment the therapy-induced enteropathy is steroid-refractory, particularly steroid-refractory ICI-induced enteropathy. However, in another embodiment, the subject is steroid-naïve. In an still further embodiment, the subject is steroid-naïve, and exhibits grade 1-2 therapy-induced colitis according to CTCAE.

In another embodiment, the engineered LAB are administered or used (or for use) in conjunction with a steroid.

The ICI therapy may be therapy with any immune checkpoint inhibitor. An immune checkpoint inhibitor is broadly defined as any agent which inhibits the activity or function of a checkpoint protein. This may be an agent which binds to a checkpoint protein or to a receptor for a checkpoint protein. A checkpoint inhibitor may thus be a binding agent for a checkpoint protein or for a receptor therefor. A binding agent may be, or may be based on or derived from, an antibody. The antibody may be a natural or synthetic antibody, or a fragment or derivative thereof. The three principal immune checkpoints which are targeted today by ICIs are PDL-1, PD-L1 (PD-L1 is Programme Death Ligand 1 and PD-1 is the receptor for PD-L1), and CTLA-4 (cytotoxic T-lymphocyte antigen-4), but the medical uses and methods herein are not limited to these, and include targeting of any immune checkpoint.

Other checkpoint proteins include CD-137 (4-1BB) which is a costimulatory checkpoint protein; lymphocyte activation gene 3 (LAG-3, CD223), a CD4-related inhibitory receptor co-expressed with PD-1 on tolerant T cells; B7 superfamily proteins B7-H3 and B7-H4; T cell protein TIM3; and phosphatidylserine (PS) which is a phospholipid in normal cells that is translocated to the outer member surface during apoptosis, suppressing the excess immune activation that would otherwise occur during processing and clearance of decaying cell matter.

Examples of checkpoint inhibitors include: Tremelimumab (CP-675,206), a human IgG2 monoclonal antibody with high affinity to CTLA-4; Ipilimumab (MDX-010), a human IgG1 monoclonal antibody to CTLA-4; Nivolumab (BMS-936558), a human monoclonal anti-PD1 IgG4 antibody that essentially lacks detectable antibody-dependent cellular cytotoxicity (ADCC); MK-3475 (formerly lambrolizumab), a humanized IgG4 anti-PD-1 antibody that contains a mutation at C228P designed to prevent Fc-mediated ADCC;

Urelumab (BMS-663513), a fully human IgG4 monoclonal anti-CD137 antibody; anti-LAG-3 monoclonal antibody (BMS-986016); and Bavituximab (chimeric 3G4), a chimeric IgG3 antibody against PS; MPDL3280A (RG7446), a human IgG1-kappa anti-PD-L1 monoclonal antibody; and MEDI4736, another IgG1-kappa PD-L1 inhibitor.

Another alternative approach is to competitively block the PD-1 receptor, using a B7-DC-Fc fusion protein, and such fusion proteins can also therefore be used.

Accordingly, in an embodiment the immune checkpoint inhibitor is an antibody against PDL-1, PD-1, CTLA4, TIM3, CD137, CD223, PS, or a KIR on an NK cell, or it is B7-DC-Fc fusion protein.

The LAB for use herein are engineered to express a wound-healing and/or inflammation-resolving protein. One or more wound healing and/or inflammation-resolving proteins may be expressed. The term “engineered” means that the LAB have been modified to express the protein, more particularly genetically-modified. Thus, the term “engineered” is synonymous with, and may be used interchangeably with “modified” or “genetically modified”. In other words, the engineered LAB are LAB into which have been introduced one or more nucleic acid molecules comprising a nucleotide sequence encoding the protein. The introduced nucleic acid molecule thus encodes a protein which is heterologous to the LAB (i.e. not natively expressed). The term “nucleotide sequence” is used herein synonymously and interchangeably with “gene” or “gene sequence” to refer to a sequence encoding the protein in question. In particular, the use of the term “gene” herein does not imply or require the presence with the coding sequence of any promoter sequence or other expression control sequence. Thus, the term “gene” does not imply or require that the native promoter or other control sequence of the native gene is present, merely a coding sequence encoding the stated protein.

The nucleic acid molecule may be introduced into the LAB in, or as part of an autonomously replicating element, e.g. a plasmid as described further below, or another vector, or it may be integrated into the chromosome of the recipient, or host, LAB. Thus, the nucleotide sequence encoding the protein may be present in the engineered LAB integrated in the host genome, or independent of the host genome, in a vector that is present in the engineered LAB.

The wound-healing protein may be any protein which has an effect of promoting healing of a wound. Similarly, the inflammation-resolving protein may be any protein which has an effect in promoting or aiding the resolution of inflammation. In other words, the inflammation-resolving protein may be referred to as an “anti-inflammatory” protein. In particular, the wound-healing and/or inflammation-resolving protein is a mammalian protein. The wound healing and/or inflammation-resolving protein may advantageously be an immunomodulatory protein, that is a protein which has an effect in modulating the activity of immune cells. Thus, in an embodiment, the wound healing and/or inflammation-resolving protein may be defined as an immunoactive protein, in particular an immunoactive protein which active locally on immune cells present in the vicinity of the wound, in this case the enteropathy, i.e. locally in the gut of the subject. In a particular embodiment. the protein is an anti-inflammatory protein, or a protein with anti-inflammatory activity. In particular, the protein may act to stimulate the growth and/or activity of immune cells, particularly immune cells present in the gut, and more particularly macrophage cells. The protein may act to promote or increase the anti-inflammatory effect of the immune cells (e.g. macrophages). The protein may stimulate the proliferation of local macrophages and/or other immune cells and may induce a phenotypic shift to an anti-inflammatory phenotype. Alternatively, or additionally the protein may have anti-fibrotic effects, i.e. it may be an anti-fibrotic protein.

The wound healing and/or inflammation-resolving protein may have an effect in ameliorating, that is improving or reducing one or more of the clinical signs or symptoms or histological features of therapy-induced enteropathy as discussed above. In particular, the protein may be seen to reverse disease progression, thus, for example, the protein may act to reduce crypt damage and epithelial loss, or apoptosis of cells in the gut, for example as determined histologically, to reduce inflammatory markers, to reduce colon shortening etc., or to reduce any marker of disease activity. The protein may further act to inhibit (i.e. to prevent or reduce) the development of fibrosis in the GI tract, or more particularly in the intestine.

The protein may be a cytokine, e.g. an interleukin, or a chemokine, or a growth factor. It may be a CXC protein. In an embodiment the protein is selected from one or more of CXCL12, CXCL17, Ym1, TGF-β, IL-22, IL-27 IL-4, IL-10, IL-12, IL-8, or SP1. In a more particular embodiment the protein is selected from one or more of CXCL12, CXCL17, Ym1 or TGF-β

CXCL12 (also known as SDF-1; SEQ ID NO: 3 and 6) is constitutively expressed in tissues and acts through the receptor CXCR4 found on leukocytes and endothelial cells inducing multiple cellular actions. CXCL12 is found in high levels in macrophages specialized in tissue remodeling.

CXCL17 (SEQ ID NO: 9 and 12), originally classified as a chemokine, has similar effects on the phenotype of tissue macrophages as CXCL12. In similarity with CXCL12, CXCL17 is co-regulated with VEGF-A measured in cell culture. CXCL17 is found mainly in mucosal tissues and have been reported to be directly antimicrobial to pathogenic bacteria that are also found on skin whilst showing no effect on survival of Lactobacillus casei. As well as anti-microbial effects, it has been reported to have microbial and anti-fibrotic effects and chemotactic properties. More recently, the classification of CXCL17 as a chemokine has been questioned, but this is not relevant to its proposed use herein.

A further protein of interest is Ym1 (SEQ ID NO: 15 and 18), which is a chitinase-like protein. Chitin is a common polysaccharide in bacterial biofilm. Ym1 both counteracts biofilm production and induces macrophage functions important for tissue remodeling and wound healing and is specific to macrophages since it is not taken up by either vascular cells or epithelial cells.

Another protein of interest is TGF-β. TGF-β occurs in three different isoforms, TGF-β 1, 2 and 3, all of which are included herein. TGF-β is a multifunctional cytokine, and is secreted by many cell types, including macrophages, and plays a role in the regulation of inflammatory processes, including in the gut.

More specifically, the protein may be murine CXCL12, in particular murine CXCL12-1α (SEQ ID NO: 3); human CXCL12, in particular human CXCL12-1α (SEQ ID NO: 6); murine CXCL17 (SEQ ID NO: 9); human CXCL17 (SEQ ID NO: 12); murine Ym1 (SEQ ID NO: 15); human Ym1 (SEQ ID NO: 18) and human TGF-β (SEQ ID NO. 27).

In one embodiment, the protein is selected from murine CXCL12-1α having an amino acid sequence as shown in SEQ ID NO: 3 or 2, or an amino acid sequence with at least 80% sequence identity thereto; human CXCL12-1α having an amino acid sequence as shown in SEQ ID NO: 6 or 5, or an amino acid sequence with at least 80% sequence identity thereto;

murine CXCL17 having an amino acid sequence as shown in SEQ ID NO: 9 or 8, or an amino acid sequence with at least 80% sequence identity thereto; human CXCL17 having an amino acid sequence as shown in SEQ ID NO: 12 or 11, or an amino acid sequence with at least 80% sequence identity thereto; murine Ym1 having an amino acid sequence as shown in SEQ ID NO: 15 or 14, or an amino acid sequence with at least 80% sequence identity thereto; human Ym1 as shown in SEQ ID NO: 18 or 17 or an amino acid sequence with at least 80% sequence identity thereto; and human TGF-β as shown in SEQ ID NO: 27 or an amino acid sequence with at least 80% sequence identity thereto.

A nucleic acid molecule comprising a nucleotide sequence encoding a protein as defined or described above may be prepared. The nucleic acid molecule may be provided in the form of a recombinant construct comprising the coding nucleotide sequence(s) operably linked to one or more expression control sequence, for example a promoter, optionally with one or more further regulatory sequences. Each coding sequence may be under the control of a separate expression control sequence, but for convenience, all the coding sequences to be introduced may be under the control of the same expression control sequence(s). A construct comprising a nucleic acid molecule comprising one or more coding sequences and one or more expression control sequences may be referred to herein as an expression construct.

The nucleic acid molecule or recombinant construct may be comprised within a vector, e.g. for the purposes of cloning, or for expression, e.g. in an expression vector. As used herein, the term “vector” refers to any genetic element capable of serving as a vehicle of genetic transfer, expression, or replication for an exogenous nucleic acid sequence in a host strain. A vector may exist as a single nucleic acid molecule or as two or more separate nucleic acid molecules. Vectors may be single copy vectors or multicopy vectors when present in a host LAB

A particular vector for use herein is an expression vector. In such a vector, one or more genes can be inserted into the vector molecule, in proper orientation and proximity to expression control elements resident in the expression vector molecule so as to direct expression of one or more proteins when the vector molecule is present in the host LAB.

Construction of appropriate expression vectors and other recombinant or genetic modification techniques for practising the method herein are well known in the art (see, e.g., Green and Sambrook, Molecular Cloning, A Laboratory Manual, Cold Spring Harbor Laboratory Press (Cold Spring Harbor, N.Y.) (2012), and Ausubel et al., Short Protocols in Molecular Biology, Current Protocols John Wiley and Sons (New Jersey) (2002).

The vector can be a plasmid, cosmid, phagemid or other phage vector, viral vector, episome, an artificial chromosome, e.g. bacterial artificial chromosome (BAC) or P1 artificial chromosome (PAC), or other polynucleotide construct, and may, for example, include one or more selectable marker genes and appropriate expression control sequences.

Generally, regulatory control sequences are operably linked to the coding nucleic acid sequences, and include constitutive, regulatory and inducible promoters, transcription enhancers, transcription terminators, and the like which are well known in the art. As noted above, the coding nucleic acid sequences can be operably linked to one common expression control sequence or linked to different expression control sequences.

In particular embodiment, the nucleotide sequence(s) is/are provided in a plasmid. More particularly, the plasmid is for use in expressing a protein in lactic acid bacteria and is accordingly provided, or adapted, for such use (e.g. it is designed, selected, adapted or modified for specific or particular use in lactic acid bacteria). Thus, in one embodiment the plasmid is for specific expression in lactic acid bacteria, as compared to bacteria or microorganisms generally. The plasmid may be adapted for expression in lactic acid bacteria by means of its regulatory elements (regulatory sequences) and/or coding sequences, e.g. which are selected or modified for expression in lactic acid bacteria.

Accordingly, the plasmid may comprise one or more regulatory (i.e. expression control) sequences which permit expression, or which are specific for expression, in lactic acid bacteria. Thus, the plasmid may contain expression control sequences derived from, or suitable for, or specific for, expression in lactic acid bacteria. Appropriate expression control sequences include for example translational (e.g. start and stop codons, ribosomal binding sites) and transcriptional control elements (e.g. promoter-operator regions, termination stop sequences), linked in matching reading frame with the nucleotide sequence(s) which encode the protein(s) to be expressed. The regulatory sequences(s) are operably linked to a nucleotide sequence encoding said protein, such that they drive, or control, expression of the protein.

The plasmid may be introduced into a lactic acid bacterial cell by any suitable transformation technique, and such techniques are well described in the literature. The bacterial cell may be cultured or otherwise maintained under conditions permitting expression of said protein from the plasmid. This may include conditions in the GI tract in a subject.

In one embodiment the promoter in the plasmid which controls expression of the protein is a promoter which permits, or which is specific for, expression in lactic acid bacteria. Thus, the plasmid may comprise a nucleotide sequence(s) encoding the protein(s), under the control of (or operably linked to) a promoter capable of expressing the protein in lactic acid bacteria. In a particular preferred embodiment, the plasmid comprises a lactic acid bacteria promoter, that is the promoter which controls expression of the protein(s) is a promoter which is derived from a lactic acid bacterium, or more particularly which is obtained or derived from a gene expressed in a lactic acid bacterium.

In some embodiments, in addition to a lactic acid bacterial promoter, the plasmid may also contain other regulatory elements or sequences obtained or derived from lactic acid bacteria to control expression of the protein(s). Thus, for example such other lactic acid bacterial expression control elements or sequences may include enhancers, terminators and/or translational control elements or sequences as discussed above. In some embodiments the plasmid may also contain regulatory elements or sequences which control or regulate expression from the promoter e.g. operator sequences etc. or one or more regulatory genes, as discussed further below.

Alternatively, or additionally, the plasmid may be adapted (or modified etc.) for use in lactic acid bacteria by virtue of the nucleotide sequences encoding the protein(s) being codon-optimised for expression in lactic acid bacteria.

In a preferred embodiment the promoter for expression of the protein is a regulated (regulatable) or inducible promoter. Thus, expression of the protein may be controlled or regulated (e.g. initiated, for example at a desired or appropriate time) by providing or contacting the bacteria with a regulatory molecule or inducer which activates or turns on (induces) the promoter. This is advantageous in the context of delivery of the protein to the site of enteropathy.

An expression system for expressing the protein in lactic acid bacteria may comprise (i) a plasmid comprising a nucleotide sequence encoding the protein under the control of an inducible promoter capable of expressing the protein in lactic acid bacteria; and (ii) an inducer (or regulatory molecule) for the promoter. The expression system may conveniently be provided in the form of a kit comprising components (i) and (ii) above.

Accordingly, in a particular aspect there is provided a pharmaceutical product (e.g. a kit or combination product) comprising:
lactic acid bacteria comprising a nucleotide sequence encoding a said protein
p under the control of an inducible promoter capable of expressing the protein in lactic acid bacteria; and
an inducer (or regulatory molecule) for the promoter
as a combined preparation for separate, sequential or simultaneous use in treating or preventing therapy-induced enteropathy in a subject.

Generally speaking, in any aspect herein the subject may be any human or animal subject, including for example domestic animals, livestock animals, laboratory animals, sports animals or zoo animals. The animal is particularly a mammalian animal, but other animals, e.g. birds are included. Thus, the animal may be a primate, a rodent (e.g. a mouse or rat), or a horse, dog or cat. Most preferably the subject is a human.

Lactic acid bacteria (LAB) or Lactobacillies are a clade of Gram-positive, low-GC, acid-tolerant, generally nonsporulating, non-respiring, either rod-shaped (bacillus), or spherical (coccus) bacteria which share common metabolic and physiological characteristics. These bacteria produce lactic acid as the major metabolic end product of carbohydrate fermentation and are characterized by an increased tolerance to acidity (low pH range). These characteristics of LAB allow them to outcompete other bacteria in a natural fermentation because LAB can withstand the increased acidity from organic acid production (e.g. lactic acid). Thus, LAB play an important role in food fermentations, as acidification inhibits the growth of spoilage agents. Several LAB strains also produce proteinaceous bacteriocins which further inhibit spoilage and growth of pathogenic microorganisms. LAB have generally recognized safe (GRAS) status and are amongst the most important groups of microorganisms used in the food industry.

The core genera that comprise the lactic acid bacteria group are Lactobacillus*, Leuconostoc, Pediococcus, Lactococcus, Enterococcus, Weissella, and Streptococcus, as well as the more peripheral Aerococcus, Carnobacterium, Enterococcus, Oenococcus, Sporolactobacillus, Tetragenococcus, and Vagococcus. Any lactic acid bacterium from these genera may be used in the methods herein, but particularly bacteria from the genera Lactobacillus or Lactococcus, and more particularly from Lactobacillus. (*Lactobacillus, Pediococcus, Weissella and Leuconostoc have recently been unified but together also been divided into 25 genera. For the sake of simplicity, herein the old taxonomic classification is used. Reference: Zheng, J., Wittouck, S., Salvetti, E., Franz, C. M. A. P., Harris, H. M. B.,

Mattarelli, P., et al. (2020). A taxonomic note on the genus Lactobacillus: Description of 23 novel genera, amended description of the genus Lactobacillus Beijerinck 1901, and union of Lactobacilloae and Leuconostocaceae. International Journal of Systematic and Evolutionary Microbiology, 70(4), 2782-2858. http://doi.org/10.1099/ijsem.0.004107).

In one embodiment the LAB is a Lactobacillus. This may include any species of Lactobacillus, a wide range of which are known, and many of which have been deposited and are publically available from culture deposit institutions. More particularly, the Lactobacillus is Lactobacillus reuteri. This species has recently been reclassified as Limosilactobacillus reuteri. Thus, the term “Lactobacillus” as used herein includes Limosilactobacillus (or alternatively put, in an embodiment, the LAB is a Lactobacillus or a Limosilactobacillus).

Lactobacillus reuteri may of any strain, a number of which are known and reported in the art. Many strains of Lactobacillus reuteri are publicly available from culture collections, for example, or Lactobacillus reuteri DSM20016 or Lactobacillus reuteri ATCC PTA 6475). Particular mention may be made of the strain Lactobacillus reuteri R2LC.

Lactobacillus reuteri R2LC has been reported in the literature and is available

on request from Prof. Siv Ahnre, Lund University, Sweden (see Ahnre et al., Nutrients 2011, 3, 104-117). Lactobacillus reuteri strain R2LC has been deposited at the Culture Collection of the University of Gothenburg (CCUG) in December 2021 with the preliminary deposit number R2LC20211221.Lactobacillus reuteri strain R2LC has been deposited under the terms of the Budapest Treaty at the Leibniz Institute DSMZ-German Collection of Microorganisms and Cell Cultures (Inhoffenstraβe 7 B, D-38124 Braunschweig, Germany) on 26 Aug. 2022 with the accession number DSM 34372.

As noted above, the LAB may comprise or express one or more of said proteins. Thus, it may comprise or express a combination of two or more of any of the proteins listed above, for example a combination of a CXCL12, CXCL17, Ym1 and/or TGF-β protein (e.g. 2 or more of CXCL12, CXCL17, Ym1 or TGF-β). Alternatively, it may comprise or express 2 or more types of a CXCL12, CXCL17, Ym1 and/or TGF-β) protein, or indeed of any other protein listed or mentioned above, (e.g. both murine and human CXCL12 etc.). Where more than one protein is expressed, the protein may be expressed from a nucleotide sequence encoding the proteins under the control of a single promoter, or more than one promoter may be used. For example, each protein may be expressed from a separate promoter, which may be the same or different. Techniques for expression of 2 or more proteins together from the same plasmid are well known in the art and include for example translational coupling techniques etc., means for achieving this are known and available in the art. For example, multiple transgenes can be expressed simultaneously under one promoter using P2A and T2A sequences.

The protein may be a native or natural protein (i.e. the nucleotide sequence may encode a protein having an amino acid sequence as found in nature) and may be from any species in which these proteins occur. Generally, the protein will be a mammalian protein and as indicated above human and murine proteins are preferred. However, the native nucleotide sequences or protein sequences may be modified, for example by one or more amino acid additions, insertions, deletions and/or substitutions, as long as the function or activity of the protein is not substantially or significantly altered, e.g. as long as the activity of the protein is substantially retained. The protein may be a fragment or truncated variant of a natural protein. For example, a sequence-modified variant protein may exhibit at least 80, 85, 90 or 95% of the activity of the parent protein from which it is derived. This may be assessed according to tests known in the art for activity of the protein in question. For example, activity can be measured in systems of receptor phosphorylation or calcium flux upon ligation in culture cells treated with the protein, in systems of cell chemotaxis in vitro or in vivo in models of cell recruitment to the infected protein. An in vitro assay based on chemotaxis is described in Nesmelova et al., JBC Papers in Press, Jun. 12, 2008, DOI 10.1074/jbc.M803308200 or Massena et al., Blood. 2015 Oct. 22;12 (17): 2016-26. doi: 10.1182/blood-2015-03-631572.

Epub 2015 Aug. 18. Hatse et al., Cytometry A. 2004 Oct;61(2): 178-88 describes a further in vitro chemokine activity test which might be used. The terms “CXCL12”, “CXCL17”, “Ym1” or “TGF-β” or any of the other protein names or acronyms mentioned above include not only the native proteins but also functionally equivalent variants or derivatives thereof. The proteins may thus be synthetic or sequence-modified variants, or may comprise one or more other modifications, e.g. post-translational modifications etc.

As mentioned above, the encoded proteins may have the amino acid sequences indicated above for the native human or murine proteins, namely SEQ ID NOS. 3 and 6 for murine and human CXCL12 respectively, 9 and 12 for murine and human CXCL17 respectively, and 15 and 18 for murine and human Ym1 respectively or SEQ ID NO. 27 for human TGF-β, or an amino acid sequence having at least 80% sequence identity to any aforesaid sequence. Advantageously, as further indicated above, the nucleotide sequences encoding these native proteins may be codon-optimised for expression in lactic acid bacteria. This may result in a modified amino acid sequence of the protein encoded. For example, codon-optimised sequences may encode sequences such as secretion sequences suitable, (or better suited) for lactic acid bacteria. Thus, the “optimized” protein encoded by a codon-optimised nucleotide sequence may include an altered or substituted leader or signal sequence (e.g. secretory sequence) as compared to the native protein. In a preferred embodiment the mature or cleaved form of the protein encoded by the codon-optimised sequence is identical to the native protein. Proteins encoded by codon-optimised nucleotide sequences may have an amino acid sequence as shown in SEQ ID NOS. 2, 5, 8, 11, 14, or 17. Thus, the protein encoded by the plasmid may have an amino acid sequence as shown in SEQ ID NOS. 2 and 5 for murine and human CXCL12 respectively, 8 and 11 for murine and human CXCL17 respectively, and 14 and 17 for murine and human Ym1 respectively, or an amino acid sequence having at least 80% sequence identity to any aforesaid sequence.

In other embodiments the encoded protein(s) may have an amino acid sequence which has at least 81%, 82%, 83%, 84%, 85%, 86%, 87%, 88%, 89% 90%, 91% 92% 93%, 94%, 95%, 96%, 97%, 98% or 99% sequence identity with any aforesaid amino acid sequence.

Sequence identity may readily be determined by methods and software known and readily available in the art. Thus, sequence identity may be assessed by any convenient method. However, for determining the degree of sequence identity between sequences, computer programs that make multiple alignments of sequences are useful, for instance Clustal W (Thompson et al., 1994) Nucleic Acids Res., 22:4673-4680.

Programs that compare and align pairs of sequences, like ALIGN (Myers and Miller (1988) CABIOS, 4:11-1), FASTA (Pearson and D. J. Lipman 1988 PNAS, 85:2444-2448; and Pearson (1990), Methods Enzymol., 183:63-7), BLAST and gapped BLAST

(Altschul et al., (1997). Nucleic Acids Res., 25:3389-3402) are also useful for this purpose, and may be used using default settings. Furthermore, the Dali server at the European Bioinformatics institute offers structure-based alignments of protein sequences. Multiple sequence alignments and percent identity calculations may be determined using the standard BLAST parameters, (e.g. using sequences from all organisms available, matrix Blosum 62, gap costs: existence 11, extension 1). Alternatively, the following program and parameters may be used: Program: Align Plus 4, version 4.10 (Sci Ed Central Clone Manager Professional Suite). DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix.

Variants of the naturally occurring polypeptide sequences as defined herein can be generated synthetically e.g. by using standard molecular biology techniques that are known in the art, for example standard mutagenesis techniques such as site-directed or random mutagenesis (e.g. using gene shuffling or error prone PCR).

Derivatives of the proteins as defined herein may also be encoded. By derivative is meant a protein as described above or a variant thereof in which the amino acid is chemically modified e.g. by glycosylation and such like etc.

Where a protein comprises an amino acid substitution relative to the sequence of the native protein, the substitution may preferably be a conservative substitution. The term “a conservative amino acid substitution” refers to any amino acid substitution in which an amino acid is replaced (substituted) with an amino acid having similar physicochemical properties, i.e. an amino acid of the same class/group. For instance, small residues Glycine (G), Alanine (A) Serine(S) or Threonine (T); hydrophobic or aliphatic residues Leucine (L), Isoleucine (I); Valine (V) or Methionine (M); hydrophilic residues Asparagine (N) and Glutamine (Q); acidic residues Aspartic acid (D) and Glutamic acid (E); positively-charged (basic) residues Arginine (R), Lysine (K) or Histidine (H); or aromatic residues Phenylalanine (F), Tyrosine (Y) and Tryptophan (W), may be substituted interchangeably without substantially altering the function or activity of the protein.

As indicated above, it is advantageous to use an inducible promoter for expression of the protein. By “inducible” is meant any promoter whose function (i.e. activity, or effect in allowing or causing transcription of the coding nucleotide sequence) can be regulated or controlled. The term “inducible” is thus synonymous, and may be used interchangeably with “regulatable” (or “regulated”). Thus, there is not constitutive expression of the protein. Accordingly, expression of the protein may be induced, or turned on (or more particularly turned on and off). More particularly, expression may be induced, or turned on for a finite or defined time. This may be because expression ceases after a period of time, and/or because the bacterial cells die.

In some embodiments there may be no expression (transcription) from the promoter until the promoter is induced (or alternatively termed, activated). However, as with any biological system, lack of activity may not be absolute and there may be some basal promoter activity in the absence of promoter activation or induction. However, in a preferred embodiment any basal expression of the uninduced promoter is low, minimal, or insignificant, or more preferably de minimis or negligible. Thus, expression from the inducible promoter is advantageously measurably or demonstrably increased when the promoter is induced compared to the promoter when it is not induced.

Inducible promoters are well known in the art, including inducible promoters for use in lactic acid bacteria and any appropriate inducible promoter may be used, suitable for expression in lactic acid bacteria.

An inducible promoter may be induced (or activated) in the presence of an inducer or activator molecule, which may act directly or indirectly on the promoter, and which may be added to induce the promoter, or more generally to cause or enable induction or activation of the promoter, and permit expression of the protein, or it may be induced (or activated) by a change in conditions of the bacteria containing the plasmid, e.g. by introducing a change of conditions to the lactic acid bacteria, e.g. starvation or depletion of a particular nutrient. An inducer of the promoter may be encoded by a regulatory gene, which in an embodiment may itself be induced or activated. The term “inducer” is thus used broadly herein to include any regulatory molecule, or indeed any permissive condition, which may activate or turn on an inducible promoter, or allow or cause an inducible promoter to be induced. Thus, induction of an inducible promoter may comprise the introduction of (e.g. contacting the lactic acid bacteria containing the plasmid with) a regulatory molecule or of a condition permissive to promoter induction (activation). In some embodiments the inducer can be an activation peptide. This may act directly, or indirectly to induce the promoter, for example as described further below.

As noted above, promoters obtained or derived from lactic acid bacteria are particularly suitable. These may be native promoters or modified or mutant promoters. A suitable promoter may for example be identified by growing lactic acid bacteria in a wound, and by determining which genes are expressed, or upregulated in the bacteria in the wound. The promoters from such genes may then be identified. Alternatively, a number of different promoters and expression systems in or for use in lactic acid bacteria have been identified and described or available in the art, including expression plasmids containing such promoters or expression systems for use in LAB. Any such known plasmid or expression system may be used as the basis for the recombinant plasmid of the invention.

Various inducible expression systems are known in the art for use with LAB such as Lactobacilli. One example includes an auto-inducing system based on the manganese starvation-inducible promoter from the manganese transporter of L. plantarum NC8 as described in Böhmer et al. FEMS Microbiol Lett 342 (2013) 37-44.

This system does not require the addition of external inducers for recombinant protein production. Duong et al. Microbial Biotechnology (2010) 4 (3), 357-367 describe expression

vectors for use with lactobacilli based on the broad range pWV01 replicon and containing promoters from operons involved in fructooligosaccharide (FOS), lactose or trehalose metabolism or transport, or in glycolysis. Such promoters may be induced by their specific carbohydrate and repressed by glucose.

More particularly, the inducible expression system may comprise inducible promoters involved in the production of LAB proteins, and in particular bacteriocins. The activity of such promoters may be controlled by a cognate regulatory system based on the bacteriocin regulon, for example a two-component regulatory (signal transduction) system which responds to an externally added activator peptide (i.e. an inducer/regulatory molecule in peptide form) and genes encoding a histidine protein kinase and response regulator necessary to activate this promoter upon induction by an activator peptide.

In an embodiment the expression system may be based on the nisin-controlled expression (NICE) system, based on the combination of the nisA promoter and the nisRK regulatory genes. This system is based on the promoters and regulatory genes from the Lactococcus lactis nisin gene cluster and has been used to develop regulated gene expression systems for lactococci, lactobacilli and other Gram-positive bacteria (reviewed briefly in Sørvig et al., FEMS Microbiol Lett. 2003; 229(1):119-126, and Sørvig et al., Microbiology. 2005July;151(Pt 7):2439-49).

Whilst the NICE systems are efficient and well-regulated in Lactococci, these systems can exhibit significant basal activity. This can be circumvented by integrating the histidine kinase and response regulator genes in the chromosome, limiting the expression systems to specially designed host strains.

In another embodiment the expression system may be based on the genes and promoter involved in the production of class II bacteriocins sakacin A (sap genes) by the sakacin A regulon or sakacin P (spp genes) by the sakacin P regulon. Such vectors are known as pSIP vectors and contain a promoter element derived from either the sakacin A or the sakacin P structural gene with an engineered Ncol site for translational fusion cloning. A variety of such vectors containing different promoters from the regulons and/or different replicons are described the Sorvig et al. papers mentioned above and any of these vectors could be used as the basis for the recombinant plasmid of the invention.

In a representative embodiment the promoter may be the P_{sapA}, P_{sppA} or P_{orfX} promoter from the sakacin A or P regulon, together with its associated or cognate regulatory genes.

In a particularly preferred embodiment, the plasmid contains the pSH71 replicon, the P_{orfX} promoter from the sakacin P regulon and the cognate regulatory genes, based on the vector pSIP411 as described in Sorvig et al., 2005. Plasmid pSIP411 is designated pLAB112 in the present application. The inducer for use in such an embodiment is preferably an activation peptide based on the peptide SppIP, e.g. an activation peptide having the sequence of SEQ ID NO.19, or an amino acid sequence with at least 80% (or more particularly at least 85, 90 or 95) sequence identity thereto. In a particular embodiment the plasmid is derived from the plasmid designated pLAB112 having the nucleotide sequence shown in SEQ ID NO: 20 (the corresponding amino acid translation is shown in SEQ ID NO: 34).

The use of an inducible promoter (or inducible expression system) may provide the advantage of a more controlled, and in particular prolonged expression of the protein in the setting of the gut i.e. when the bacteria are administered to the subject. For a better effect in treating or preventing therapy-induced enteropathy it is advantageous for the protein to be expressed by the bacteria for a period of time at the site of the enteropathy, e.g. for at least 40, 45, 50, 55 or 60 minutes, notably for at least one hour, or more. Thus, the protein may be expressed for a finite, a defined or a prolonged period of time. Results reported in WO2016/102660 show that the protein may be expressed for a period of about an hour at a wound surface. The bacteria may in some embodiments be optimised to allow expression of the protein for 2, 3 or 4 hours or more.

Continuous expression and delivery of the protein is thus desirable and this may be afforded by using the engineered LAB. By “continuous” or “prolonged” is meant that there is expression, and hence delivery, of the protein over a period of time e.g. over a period of at least an hour (or so, as discussed above). In particular this allows the protein to be effective over a period of time which is increased as compared to administration of the protein directly (i.e. as a protein product rather than by expression by the bacteria).

As discussed above, the nucleotide sequences encoding the protein(s) may be codon-optimised for expression in LAB. Accordingly, in preferred embodiments the nucleotide sequences which encode the proteins may be selected from the codon-optimised nucleotide sequences shown in SEQ ID NOS. 1, 4, 7, 10, 13 and 16 which encode murine CXCL12, human CXCL12, murine CXCL17, human CXCL17, murine Ym1 and human Ym1 respectively, or a nucleotide sequence having at least 80% sequence identity therewith.

Thus, in representative embodiments the recombinant plasmid may be chosen from the group consisting of the plasmids designated mLrCK1, comprising a nucleotide sequence as shown in SEQ ID NO: 1 (the corresponding amino acid translation is shown in SEQ ID NO: 28); mLrCK1.4, comprising a nucleotide sequence as shown in SEQ ID NO: 1; mLrCK1.7, comprising a nucleotide sequence as shown in SEQ ID NO: 1; hLrCK1, comprising a nucleotide sequence as shown in SEQ ID NO: 4 (the corresponding amino acid translation is shown in SEQ ID NO: 29); mLrCK2, comprising a nucleotide sequence as shown in SEQ ID NO: 7 (the corresponding amino acid translation is shown in SEQ ID NO: 30); hLrCK2, comprising a nucleotide sequence as shown in SEQ ID NO: 10 (the corresponding amino acid translation is shown in SEQ ID NO: 31); hLrMP1, comprising a nucleotide sequence as shown in SEQ ID NO: 13 (the corresponding amino acid translation is shown in SEQ ID NO: 32); and mLrMP2, comprising a nucleotide sequence as shown in SEQ ID NO: 16 (the corresponding amino acid translation is shown in SEQ ID NO: 33).

In some embodiments the protein is encoded by a nucleotide sequence which has at least 80%, 81%, 82%, 83%, 84%, 85%, 86%, 87%, 88%, 89% 90%, 91% 92% 93%, 94%, 95%, 96%, 97%, 98% or 99% sequence identity to a nucleotide sequence of the following codon optimized inserts mLrCK1 (i.e., to the nucleotide sequence of SEQ ID NO: 1), mLrCK1.4 (i.e., to the nucleotide sequence of SEQ ID NO: 1), mLrCK1.7 (i.e., to the nucleotide sequence of SEQ ID NO: 1), hLrCK1 (i.e., to the nucleotide sequence of SEQ ID NO: 4), mLrCK2 (i.e., to the nucleotide sequence of SEQ ID NO: 7), hLrCK2 (i.e., to the nucleotide sequence of SEQ ID NO: 10), hLrMP1 (i.e., to the nucleotide sequence of SEQ ID NO: 13), and mLrMP2 (i.e., to the nucleotide sequence of SEQ ID NO: 16).

Sequence identity of nucleotide molecules may be determined by methods and software known and widely available in the art, for example by FASTA Search using GCG packages, with default values and a variable pamfactor, and gap creation penalty set at 12.0 and gap extension penalty set at 4.0 with a window of 6 nucleotides.

Such sequence identity related nucleotide sequences may be functionally equivalent to the nucleotide sequence which is set forth in SEQ ID NO: 1, 4, 10, 13 or 16. Such nucleotide sequences may be considered functionally equivalent if they encode polypeptides which would be considered functional equivalents to the respective CXCL12, CXCL17 or Ym1 proteins. Particular functional equivalents are those which encode the particular proteins as set out above.

The bacteria may be administered in any convenient or desired way, e.g. orally, or by direct administration to the GI tract, e.g. rectally, or by direct injection or infusion or application or introduction of a pharmaceutical composition or dressing or device containing the bacteria. In a particular embodiment, the bacteria are administered perorally.

The bacteria may be formulated or prepared in any convenient or desired way for administration by any of the above routes, according to procedures and using means well known and routine in the art. Thus, as well as pharmaceutical compositions, medical devices and dressings etc., products for use herein may be formulated and provided as or in nutritional supplements or foods, e.g. functional foods.

Oral administration forms include powders, tablets, capsules and liquids etc. Further the bacteria may be provided on (e.g. coated on) the surface of a medical device such as an implant (e.g. a prosthetic implant), tube, line or catheter etc.

The bacteria may be provided in any convenient or desired form, e.g. as an active or growing culture or in lyophilized or freeze-dried form.

Generally speaking, for administration herein, the LAB may conveniently be provided as an enteric preparation, that is as a preparation which is protected from digestion in the stomach, and which is designed to release the LAB in the intestine. The preparation may thus be gastric juice resistant. It may be designed to release the LAB at neutral pH. The formulation of enteric preparations and compositions is known in the art and described in the literature, using for example coatings and/or excipients In particular, a composition containing the LAB may be provided with an enteric coating. Such coatings are commercially available, including for example, the enteric coatings available from Evonik Health Care under the brand name Eudragit.

In an embodiment, the LAB may be provided as or in a tablet, capsule or microparticulate which is provided with an enteric coating.

The composition comprising the LAB may also contain the inducer (where an inducible promoter is used). This may be provided as part of the product (e.g. incorporated into or included in a tablet or capsule) or separately, e.g. as part of a kit or combination product, as defined above.

When co-formulated together in a product the bacteria and the inducer may be provided in a format in which the bacteria are separated from the inducer and are brought together (or contacted) in use. For example, the bacteria and inducer may be in separate compartments which are brought together (e.g. contacted or mixed), or may be separated by a barrier (e.g. a membrane or other partition) which may be broken or disrupted or opened in use.

Alternatively, the inducer may be formulated and provided separately (e.g. in a kit also containing the bacteria, or a product containing the bacteria), and the inducer and bacteria (or product containing the bacteria) may be brought together (e.g. contacted) during use. This may be before, during or after administration to the subject. For example, a product comprising the bacteria may be administered first and then the inducer may be added or applied to the bacteria. In another embodiment the bacteria and inducer may be premixed before administration, e.g. just before or immediately before, or during administration.

Where bacteria are provided in lyophilized or freeze-dried form, it may be desirable to reconstitute, or resuspend, them prior to administration e.g. prior to or during use. However, for use herein, it is convenient to provide them in lyophilized or freeze-dried form, for reconstitution by body fluids when released in the GI tract.

Conveniently, for use herein the LAB may be formulated in enteric-coated capsules comprising lyophilized bacteria and lyophilized inducer.

Alternatively, a tablet may comprise at least two separate compartments, wherein one compartment comprises freeze-dried bacteria and the other compartment comprises the inducer, and optionally a liquid. The tablet is squeezed before ingestion so that an inner membrane, separating the two compartments, is broken and the contents are mixed together.

The engineered LAB may be used as proposed herein to treat therapy-induced enteropathy in an advantageous manner. It is believed that an improved therapeutic effect may be obtained as compared to existing treatments, and in particular compared to treatment with steroids. Indeed, therapy-induced enteropathy may become resistant, or refractory to treatment with steroids, and thus the present proposal opens up the opportunity to treat a previously intractable group of patients, e.g. those with diagnosed steroid-refractory ICI-induced colitis.

Further, an advantage afforded by the presently-proposed treatment is to spare the subject from steroid therapy.

Representative embodiments are further described with reference to the following non-limiting Examples and Figures.

### DESCRIPTION OF FIGURES

FIG. 1. Effect of ILP100 on PD-1 inhibitor induced colitis in mice. Hematoxylin and eosin-stained mice colon tissue sections (A), crypt damage scores (B) and graph of mean crypt damage scores (C). Mice were subcutaneously inoculated with breast cancer tumor cells. Treatment started when tumors reached a specific size. Group A: no treatment for 3 weeks. Group B: anti-PD-1 antibody+isotype control monoclonal antibody (mAb) for 3 weeks. Groups C: anti-PD-1 antibody isotype control mAb for 3 weeks+ILP100 treatment for 1 week (week 3). One-way ANOVA multiple comparations followed by Holm-Sidak post-hoc test. Mice/sample per group: n=4.

FIG. 2. Effect of ILP100 on PD-1 induced loss of epithelium in mice. Graph shows epithelial loss measured at termination for mice treated with isotype control, anti-PD-1 or anti-PD-1 followed by R2LC_CXCL12 One-way ANOVA multiple comparations followed by Holm-Sidak post-hoc test. Mice/sample per group: n=4-6.

FIG. 3. Effect of ILP100 on PD-1 and PDL-1 induced colon shortening in mice. Graph shows colon length (cm) in mice treated with an isotype control antibody and vehicle; a PD-1 inhibitor and vehicle; a PD-1 inhibitor and ILP100; a PD-L1 inhibitor and vehicle; or a PD-L1 inhibitor and ILP100.

FIG. 4. Effect of ILP100 on PD-1 inhibitor induced colon erosion/ulceration occurrence in mice. Graph shows occurrence of colon erosion/ulceration measured at termination for mice treated with isotype control, anti-PD-1 or anti-PD-1 followed by R2LC_CXCL1

FIG. 5. Effect of WT_R2LC and R2LC_hCXCL12 on combined DSS and PD-1 inhibitor induced colitis in mice. Graphs show Disease Activity Index (A), Body weight loss (B), Intestinal bleeding score (C), stool consistency score (D), colon length (E), colon weight (mg) (F) and the ratio between colon weight and length (G) in mice treated with either vehicle (DSS+ICI), WT_R2LC(DSS+ICI+WT_R2LC) or R2LC_hCXCL12 (DSS+ICI+R2LC_hCXCL12). Untreated (UT) group in FIGS. 5E-G is included as a reference and was not part of the statistical test.

FIG. 6. Effect of ILP100, WT_R2LC, anti-TNF and anti-α4β7 on DSS-induced colitis in mice. FIGS. 6A, B and E show Disease activity index (A), colon length (cm) (B), and occurrence of colon erosion/ulceration measured at termination (E) in healthy mice and mice treated with either DSS only (DSS), ILP100 (DSS+R2LC_CXCL12) or WT_R2LC (DSS+WT_R2LC). FIGS. 6C and D show Disease activity index (C) and colon length (cm) (D) in healthy mice and mice treated with either DSS only (DSS), ILP100 (DSS+R2LC_CXCL12), anti-TNF (DSS+anti-TNF) or anti-α4β7 (DSS+anti-α4β7). Mice/sample per group: n=22-32 for A and C; n=4 for B and D.

### EXAMPLES

### Example 1: Preparation of Lactobacillus reuteri Expressing CXCL12 (ILP100)

Lactobacillus reuteri strain R2LC was genetically modified with a plasmid containing a nucleotide sequence encoding human CXCL12 as described in Example 1 of WO 2016/102660. This modified strain is designated herein as ILP100.

Briefly, the sequence for hCXCL12 was codon-optimised for expression in LAB, and the resulting sequence (SEQ ID NO. 4) was introduced into plasmid pLAB112 (SEQ ID NO. 20), which corresponds to plasmid pSIP411 as described in Sørvig et al., Construction of vectors for inducible gene expression in Lactobacillus sakei and L plantarum. FEMS Microbiol Lett. 2003; 229(1):119-126.

The resulting plasmid, designated hLrCK1 (pLAB112 with optimized hCXCL12-1a insert) was transformed into Lactobacillus reuteri strain R2LC, and a clone positive for the construct was collected. The plasmid hLrCK1 from colonies of the positive clone was verified by sequence analysis. The positive clone is designated strain ILP100.

### Example 2: ILP100 Treatment Reduces Crypt Damage in Mice with PD-1 Inhibitor Induced Colitis

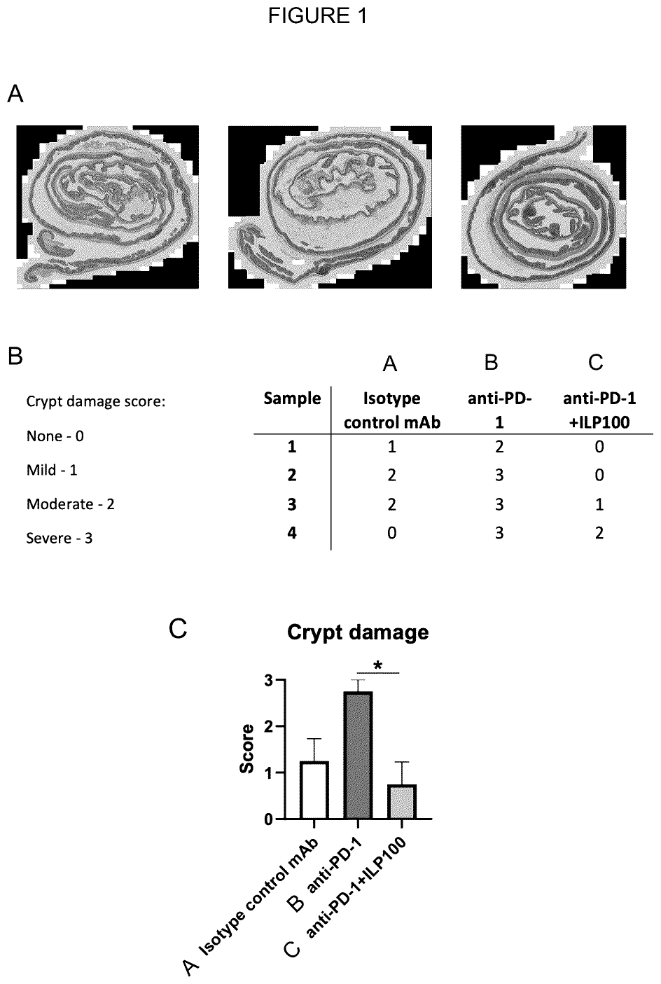

The effect of ILP100 on PD-1 inhibitor induced colitis in mice was studied in three experimental groups A, B and C (FIG. 1). On day 0, female BALB/c mice were inoculated orthotopically with 7000 4T1 cells. The tumour size in this study was monitored twice per week. The animals were treated with A) isotype control mAb weekly for 3 weeks, B) anti PD-1 antibody weekly for three weeks or C) anti PD-1 antibody weekly for three weeks and ILP100 treatment was administered orally via gavage 7 days prior to termination. At termination, a blood smear and Swiss rolls for fixation of the colon and small intestine from each animal were prepared and fixated in 4% PFA.

The Swiss rolls of colon and the small intestine tissues were embedded in paraffin, sectioned and stained with hematoxylin. The slides were scanned using a microscope

(FIG. 1A). The image files were blinded and the key was kept by one person not taking part in the image analysis or interpretation of results. Images were analysed using histopathological methods including evaluation of crypt damage. Each image was evenly divided in squares of y x y mm2 and the tissue was given a score of 0-3 (FIG. 1B). A mean value for each tissue corresponding to one animal was calculated. Then the analysis was unblinded. The mean and SEM of the groups was calculated and the results are shown in FIG. 1C.

FIG. 1C shows that Group B has a higher mean crypt damage score than Group A indicating that PD-1 treatment for 3 weeks induces significant damage to the intestine. Group C has a significantly lower mean crypt damage score compared to Group B, indicating that ILP100 treatment of PD-1 induced colitis in the mice of Group C was effective.

### Example 3: ILP100 Treatment Reduces Epithelial Loss Damage in Mice with PD-1 Inhibitor Induced Colitis

The effect of ILP100 treatment on epithelial loss in female Balb/C mice treated with PD-1 inhibitor was studied.

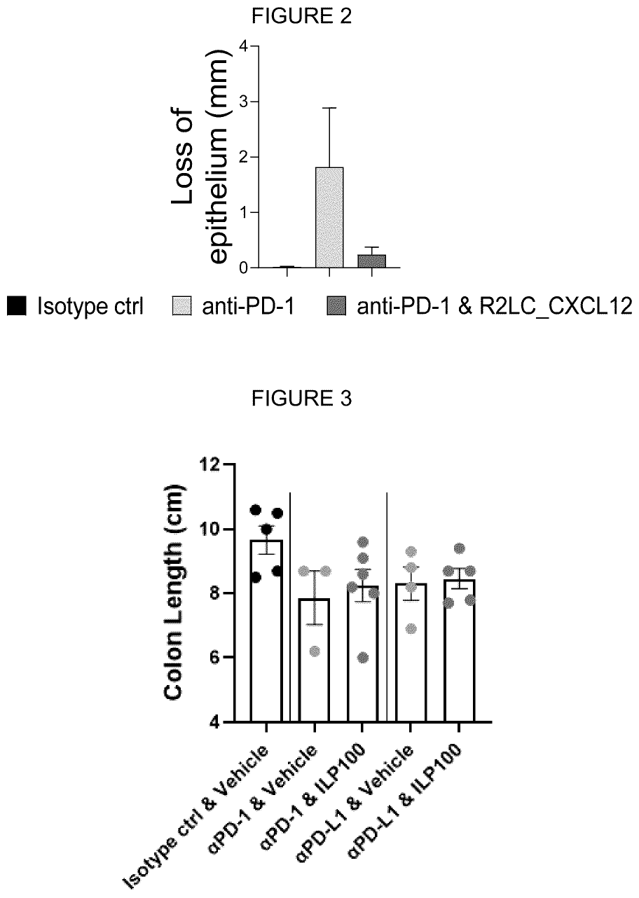

The effect of ILP100 on PD-1 inhibitor induced colitis in mice was studied in three experimental groups (FIG. 2). On day 0, mice were inoculated intraperitoneally with 7000 4T1 cells. The tumour size in this study was monitored twice per week. The animals were treated with isotype control mAb weekly for 3 weeks, anti PD-1 antibody weekly for three weeks or anti PD-1 antibody weekly for three weeks and then ILP100 treatment once per day (administered orally via gavage 7) for seven days prior to termination.

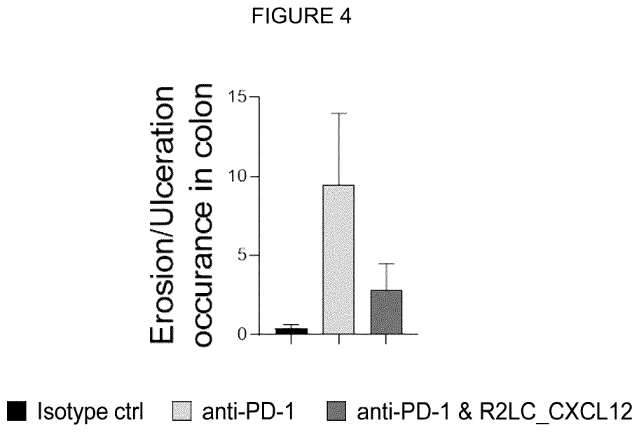

Colons were collected at the end of experiment and rolled into Swiss rolls. The tissue was paraffin embedded, sectioned, and stained with hematoxylin and eosin. The length of areas with erosion and ulceration was measured in each section. Erosion was defined as areas with no epithelium but with intact crypt structures, ulceration as areas without epithelium and no remaining crypt structure. Since there were few and very small areas of ulceration, the two parameters were merged and defined as loss of epithelium. FIG. 2 shows that the group of mice treated with PD-1 inhibitor in combination with ILP100 demonstrates less epithelial loss than mice treated with vehicle. FIG. 4 shows that mice treated with PD-1 inhibitor in combination with ILP100 demonstrates a reduced occurrence of erosion/ulceration in the colon.

Tumour size in the mice was not affected by ILP100 treatment as compared with the anti-PD1 antibody (PD1-inhibitor).

### Example 4: ILP100 Treatment Reduces Colon Shortening in Mice with PD-1 Inhibitor and PD-L1 Inhibitor Induced Colitis.

The effect of ILP100 treatment on colon length, indicative of fibrosis development, in mice treated with PD-1 inhibitor and PD-L1 inhibitor was also studied. FIG. 3 shows that the mice treated with the isotype control and vehicle had the longest colon length at the end of the experiment. In contrast, the mice treated with PD-1 inhibitor and a vehicle, or PD-L1 inhibitor and a vehicle, had significantly shorter colon lengths. Groups of mice treated with ILP100 in combination with the PD-1 inhibitor or PD-L1 inhibitor, however, demonstrated a tendency to preserved length and reduced shortening of the colon.

Further, it was observed that mice treated with anti-PD1 did not manifest traditional symptoms of colitis as measured with disease activity index, including weight loss, stool consistency, and blood in stools. However, the mice showed a severe fibrosis development (20% colon shortening), and distinct increase in erosion and ulceration, which was reverted by the ILP100 treatment, even with a short treatment time.

### Example 5: Manufacture and Coating of Capsules Comprising SppIP and ILP100

SppIP was dissolved in a solution containing 15% glucose. In order to have control of the content, the peptide solution was dispensed into well plates and lyophilized in a form similar to a tablet. The SppIP lyophilized tablet was then added into the capsules together with the lyophilized ILP100, and the capsule was then coated by drum coating using coating polymer (Eudragit L 30 D-55).

### Example 6: ILP100 Treatment Reduces Symptoms of Colitis in Mice with Combined DSS and PD-1 Inhibitor Induced Colitis

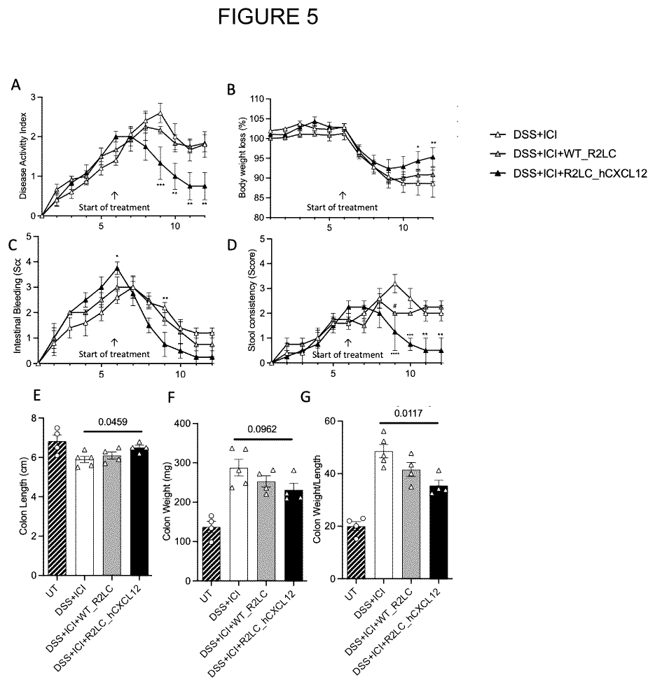

The effect of ILP100 (R2LC hCXCL12) and wild-type R2LC treatment on Disease Activity Index (DAI), body weight loss, intestinal bleeding, stool consistency, colon length, colon weight and the ratio between colon weight and length was studied in mice treated with a combination of DSS and PD-1 inhibitor (anti CTLA-4) to induce colitis. ILP100 administration during overt inflammation significantly improved colitis symptoms as evaluated by DAI compared to mice treated with either vehicle or wild-type R2LC (FIG. 5A). Furthermore, clinical symptoms of colitis (FIGS. 5 B-G) were improved in mice treated with ILP100 compared to mice treated with either vehicle or wild-type R2LC. Colon weight may be related to oedema as part of inflammation, and may be used as a marker of inflammation (the higher the weight, the more inflammation). Colon length is an indicator of fibrosis (the shorter the colon length, the greate the fibrosis and inflammation. Accordingly, the weight/length ratio is a good indicator of the induced colitis.

### Example 7: ILP100 Ameliorates DSS-Induced Colitis and Improves Symptoms with Better or Comparable Efficacy to Established Therapies for Ulcerative Colitis

Mice were treated with 3% DSS for 6 days to induce colitis. The animals were then treated with on day 6 vehicle (H₂O), WT_R2LC or ILP100 (R2LC hCXCL12) via oral gavage three times a day for 6 days or anti-TNF or anti-α4β7 via intraperitoneal injection once a day. At termination, colons were fixed and processed as described in Example 2.

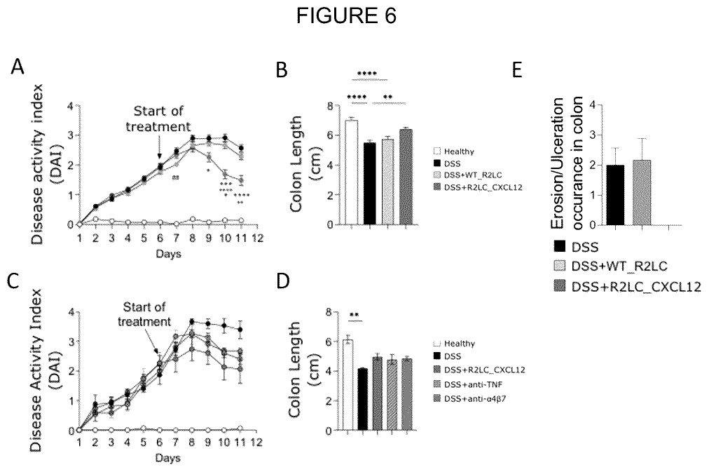

The effect of ILP100 and wild-type R2LC treatment was studied in mice with DSS-induced colitis (FIGS. 6A and B). ILP100 administration during overt inflammation significantly improved colitis symptoms as evaluated by DAI compared to vehicle treated and wild-type R2LC treated mice (FIG. 6A). Furthermore, mice treated with ILP100 had significantly longer colons compared to mice treated with vehicle or wild-type R2LC.

The effect of ILP100 treatment on DSS-induced colitis was compared to current standard of care treatments for IBD (anti-TNF and anti-α4β7; FIGS. 6C and D). ILP100 administration significantly improved colitis symptoms as evaluated by DAI with better efficacy than current standard of care treatments (FIG. 6C). Furthermore, mice treated with ILP100 had colon length comparable to mice treated with anti-TNF and anti-α4β7 treated mice (FIG. 6D). ILP100 (R2LC_hCXCL12) treated mice had the lowest DAI compared to anti-TNF and anti-α4β7 treated mice throughout the study (not significant) and were the only animals to present a significant reduction of colon shortening compared to control. Intestinal bleeding was observed to decrease more sharply in ILP100 (R2LC_hCXCL12)-treated animals compared to other treatments

The superior positive effects of the engineered LAB (ILP100 (R2LC CXCL12)) were surprising, given that the effect of the engineered bacteria occur at the local level.

### SEQUENCES

Summary of Sequence Listing

| SEQ ID NO: | Description |
|---|---|
| 1. | mLrCK1_opt DNA |
| 2. | mLrCK1_opt protein |
| 3. | mCXCL12 native protein |
| 4. | hLrCK1_opt DNA |
| 5. | hLrCK1_opt protein |
| 6. | hCXCL12 native protein |
| 7. | mLrCK2_opt DNA |
| 8. | mLrCK2_opt protein |
| 9. | mCXCL17 native protein |
| 10. | hLrCK2_opt DNA |
| 11. | hLrCK2_opt protein |
| 12. | hCXCL17 native protein |
| 13. | mYm1_opt DNA |
| 14. | mYm1 protein |
| 15. | mYm1 native protein |
| 16. | hYm1_opt DNA |
| 17. | hYm1 protein |
| 18. | hYm1 native protein |
| 19. | SppIP; activation peptide |
| 20. | pSIP411 DNA |
| 21. | pSIP411 protein |
| 22. | PCR primer |
| 23. | PCR primer |
| 24. | pVAX1 DNA |
| 25. | pCTR DNA insert |
| 26. | pCXCL12 DNA insert |
| 27. | hTGF-β |
| 28. | Amino acid translation of |
| | SEQ ID NO: 1 |
| 29. | Amino acid translation of |
| | SEQ ID NO: 4 |
| 30. | Amino acid translation of |
| | SEQ ID NO: 7 |
| 31. | Amino acid translation of |
| | SEQ ID NO: 10 |
| 32. | Amino acid translation of |
| | SEQ ID NO: 13 |
| 33. | Amino acid translation of |
| | SEQ ID NO: 16 |
| 34. | Amino acid translation of |
| | SEQ ID NO: 20 |

1. A method for treating or preventing therapy-induced enteropathy in a subject, said method comprising administering to a subject who has been or is being administered an enteropathy-inducing therapy Eengineered lactic acid bacteria (LAB) which have been engineered to express a mammalian protein which promotes resolution of inflammation and/or wound healing.

2. The method according to claim 1, wherein the inflammation resolving and/or wound-healing promoting protein is an immune-modulating protein which modulates the activity of immune cells.

3. The method according to claim 1, wherein said LAB are capable of expressing a protein selected from the group consisting of CXCL12, CXCL17 and Ym1.

4. The method according to claim 1, wherein the therapy-induced enteropathy is onco-therapy induced enteropathy.

5. The method according to claim 1, wherein the therapy-induced enteropathy is immune checkpoint inhibitor-induced (ICI-induced) enteropathy or radiation-induced enteropathy.

6. The method according to claim 1, wherein the protein is selected from:
(i) murine CXCL12-1α having an amino acid sequence as shown in SEQ ID NO: 3 or 2, or an amino acid sequence with at least 80% sequence identity thereto;
(ii) human CXCL12-1α having an amino acid sequence as shown in SEQ ID NO: 6 or 5, or an amino acid sequence with at least 80% sequence identity thereto;
(iii) murine CXCL17 having an amino acid sequence as shown in SEQ ID NO: 9 or 8, or an amino acid sequence with at least 80% sequence identity thereto;
(iv) human CXCL17 having an amino acid sequence as shown in SEQ ID NO: 12 or11, or an amino acid sequence with at least 80% sequence identity thereto;
(v) murine Ym1 having an amino acid sequence as shown in SEQ ID NO: 15 or 14, or an amino acid sequence with at least 80% sequence identity thereto; and
(vi) human Ym1 as shown in SEQ ID NO: 18 or 17 or an amino acid sequence with at least 80% sequence identity thereto.

7. The method according to claim 1, wherein the LAB are transformed with a plasmid expressing the protein.

8. The method according to claim 7, wherein the plasmid comprises one or more nucleotide sequences encoding one or more of said proteins under the control of an inducible promoter.

9. The method according to claim 7, wherein the plasmid comprises an inducible promoter and regulatory elements from the nisin regulon, the sakacin A regulon or the sakacin P regulon of a lactic acid bacterium.

10. The method according to claim 7, wherein the inducible promoter is the PorfX promoter from the sakacin P regulon.

11. The method according to claim 7, wherein the plasmid is derived from the plasmid designated pSIP411.

12. The method according to claim 1, wherein the LAB are engineered by introducing a nucleotide sequence encoding the protein which nucleotide sequence is codon-optimised for expression in lactic acid bacteria.

13. The method according to claim 1, wherein the LAB comprise one or more nucleotide sequences selected from the group consisting of: a nucleotide sequence comprising the sequence of SEQ ID NO: 1, SEQ ID NO: 4, SEQ ID NO: 7, SEQ ID NO: 10, SEQ ID NO: 13, and SEQ ID NO: 16, or a nucleotide sequence having at least 80% sequence identity to any aforesaid sequence.

14. The method according to claim 1, wherein the LAB are of the genus Lactobacillus.

15. The method according to claim 1, wherein the LAB are a strain of Lactobacillus reuteri.

16. The method according to claim 15, wherein the LAB are Lactobacillus reuteri strain R2LC.

17. The method according to claim 1, wherein LAB are provided as an orally administrable pharmaceutical composition comprising the LAB together with one or more pharmaceutically acceptable excipients.

18. The method according to claim 1, wherein the LAB are lyophilized.

19. The method according to claim 17, wherein the LAB are provided in the form of a capsule with an enteric coating.

20. The method according to claims 17, wherein the LAB express the protein under the control of an inducible promoter, and the pharmaceutical composition or capsule further comprises an inducer for the promoter.


SEQUENCE LISTING (34 sequences)

SEQ ID NO: 1 - DNA, 299 bp, Mus musculus
  source 1..299: mol_type = unassigned DNA
  CDS 3..293: protein_id = 28; translation = MAKFWKKALLTIAALTVGTSAGITSVSAKPVSLSYRCPCRFFES HIARANVKHLKILNTPNCALQIVARLKNNNRQVCIDPKLKWIQEYLEKALNK
ccatggcaaaattttggaagaaagcactattaacaattgcagccttaacagtcggcacctccgcaggaattacaagcgtttctgccaaaccggtaagtttgtcatatcgatgtccatgccggtttttcgaatctcatattgcacgcgctaatgtcaaacacttaaagattcttaatactcctaattgtgctttgcagattgttgcacgtttaaagaataacaatcgtcaagtttgtatcgatccaaagcttaaatggattcaagagtacttagaaaaggccttaaacaaataactcgag

SEQ ID NO: 2 - protein, 96 aa, Mus musculus
MAKFWKKALLTIAALTVGTSAGITSVSAKPVSLSYRCPCRFFESHIARANVKHLKILNTPNCALQIVARLKNNNRQVCIDPKLKWIQEYLEKALNK

SEQ ID NO: 3 - protein, 89 aa, Mus musculus
MDAKVVAVLALVLAALCISDGKPVSLSYRCPCRFFESHIARANVKHLKILNTPNCALQIVARLKNNNRQVCIDPKLKWIQEYLEKALNK

SEQ ID NO: 4 - DNA, 299 bp, Homo sapiens
  source 1..299: mol_type = unassigned DNA
  CDS 3..293: protein_id = 29; translation = MAKFWKKALLTIAALTVGTSAGITSVSAKPVSLSYRCPCRFFES HVARANVKHLKILNTPNCALQIVARLKNNNRQVCIDPKLKWIQEYLEKALNK
ccatggcaaaattttggaagaaagcactattaacaattgcagccttaacagtcggcacctccgcaggaattacaagcgtttctgccaaaccggtaagtttgtcatatcgatgtccatgccggtttttcgaatctcatgttgcacgcgctaatgtcaaacacttaaagattcttaatactcctaattgtgctttgcagattgttgcacgtttaaagaataacaatcgtcaagtttgtatcgatccaaagcttaaatggattcaagagtacttagaaaaggccttaaacaaataactcgag

SEQ ID NO: 5 - protein, 96 aa, Homo sapiens
MAKFWKKALLTIAALTVGTSAGITSVSAKPVSLSYRCPCRFFESHVARANVKHLKILNTPNCALQIVARLKNNNRQVCIDPKLKWIQEYLEKALNK

SEQ ID NO: 6 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 7 - DNA, 386 bp, Mus musculus
  source 1..386: mol_type = unassigned DNA
  CDS 3..380: protein_id = 30; translation = MAKFWKKALLTIAALTVGTSAGITSVSASPNPGVARSHGDQHLA PRRWLLEGGQECECKDWFLQAPKRKATAVLGPPRKQCPCDHVKGREKKNRHQKHHRKS QRPSRACQQFLKRCHLASFALPL
ccatggcaaaattttggaagaaagcactattaacaattgcagccttaacagtcggcacctccgcaggaattacaagcgtttctgccagcccaaatccaggggtcgccagatctcacggcgaccagcacctggctcctaggagatggctcctggaaggcggccaagaatgtgagtgcaaagattggttcctgcaagccccgaagagaaaagccacagctgtgctggggccaccaagaaagcagtgcccctgtgatcatgtgaagggcagagaaaagaaaaatagacaccaaaagcaccacagaaagagccaaagaccctccagagcctgccagcaatttctcaaaaggtgtcacctagccagctttgctctgcctttgtagctcgag

SEQ ID NO: 8 - protein, 125 aa, Mus musculus
MAKFWKKALLTIAALTVGTSAGITSVSASPNPGVARSHGDQHLAPRRWLLEGGQECECKDWFLQAPKRKATAVLGPPRKQCPCDHVKGREKKNRHQKHHRKSQRPSRACQQFLKRCHLASFALPL

SEQ ID NO: 9 - protein, 119 aa, Mus musculus
MKLLASPFLLLLPVMLMSMVFSSPNPGVARSHGDQHLAPRRWLLEGGQECECKDWFLQAPKRKATAVLGPPRKQCPCDHVKGREKKNRHQKHHRKSQRPSRACQQFLKRCHLASFALPL

SEQ ID NO: 10 - DNA, 386 bp, Homo sapiens
  source 1..386: mol_type = unassigned DNA
  CDS 3..380: protein_id = 31; translation = MAKFWKKALLTIAALTVGTSAGITSVSASLNPGVARGHRDRGQA SRRWLQEGGQECECKDWFLRAPRRKFMTVSGLPKKQCPCDHFKGNVKKTRHQRHHRKP NKHSRACQQFLKQCQLRSFALPL
ccatggcaaaattttggaagaaagcactattaacaattgcagccttaacagtcggcacctccgcaggaattacaagcgtttctgcctcattaaatccaggagtagcacggggtcatcgagatcggggacaagcaagtcggcgttggttacaagaaggtggtcaagaatgtgaatgtaaagattggtttttacgtgctccacgtcggaagtttatgactgttagtggtcttccaaagaaacaatgtccttgtgatcattttaagggaaatgttaagaaaactcgacaccaacgtcatcaccggaaacctaataagcattcacgggcatgtcaacaatttcttaaacaatgtcaacttcgttcttttgctcttcctctttaactcgag

SEQ ID NO: 11 - protein, 125 aa, Homo sapiens
MAKFWKKALLTIAALTVGTSAGITSVSASLNPGVARGHRDRGQASRRWLQEGGQECECKDWFLRAPRRKFMTVSGLPKKQCPCDHFKGNVKKTRHQRHHRKPNKHSRACQQFLKQCQLRSFALPL

SEQ ID NO: 12 - protein, 119 aa, Homo sapiens
MKVLISSLLLLLPLMLMSMVSSSLNPGVARGHRDRGQASRRWLQEGGQECECKDWFLRAPRRKFMTVSGLPKKQCPCDHFKGNVKKTRHQRHHRKPNKHSRACQQFLKQCQLRSFALPL

SEQ ID NO: 13 - DNA, 1226 bp, Mus musculus
  source 1..1226: mol_type = unassigned DNA
  CDS 3..1220: protein_id = 32; translation = MAKFWKKALLTIAALTVGTSAGITSVSAYQLMCYYTSWAKDRPI EGSFKPGNIDPCLCTHLIYAFAGMQNNEITYTHEQDLRDYEALNGLKDKNTELKTLLA IGGWKFGPAPFSAMVSTPQNRQIFIQSVIRFLRQYNFDGLNLDWQYPGSRGSPPKDKH LFSVLVKEMRKAFEEESVEKDIPRLLLTSTGAGIIDVIKSGYKIPELSQSLDYIQVMT YDLHDPKDGYTGENSPLYKSPYDIGKSADLNVDSIISYWKDHGAASEKLIVGFPAYGH TFILSDPSKTGIGAPTISTGPPGKYTDESGLLAYYEVCTFLNEGATEVWDAPQEVPYA YQGNEWVGYDNVRSFKLKAQWLKDNNLGGAVVWPLDMDDFSGSFCHQRHFPLTSTLKG DLNIHSASCKGPY
ccatggcaaaattttggaagaaagcactattaacaattgcagccttaacagtcggcacctccgcaggaattacaagcgtttctgcctaccaacttatgtgttactacacttcatgggctaaagatcgtccaattgaaggttcttttaagccaggaaatattgatccttgtctttgtacacatcttatttacgcttttgctggtatgcaaaataatgaaattacttacacacacgaacaagatttacgtgattacgaagctcttaatggtcttaaggataagaatactgaacttaagacacttcttgcaattggtggatggaagtttggaccagctccttttagtgcaatggtttcaactccacaaaatcggcaaatttttattcaaagtgtaattcggtttttacggcaatacaattttgatggacttaatcttgattggcaatacccaggtagtcgaggatcaccacctaaggataagcatttatttagtgttcttgtaaaagaaatgcgaaaggcttttgaagaagaaagtgttgaaaaggatattccacgtcttcttcttactagtacaggtgcaggaattattgatgtaattaagtcaggttacaagattccagaacttagtcaatcacttgattacattcaagttatgacttacgatttacacgatcctaaggatggttacacaggagaaaattctccactttacaagagtccttacgatattggaaagagtgctgatcttaatgttgattctattattagttactggaaagatcatggagctgcatcagaaaagcttattgttggttttccagcttacggacacacttttattctttcagatccatctaagacaggtattggagcacctactatttctacaggtccacctggaaagtatactgatgaaagtggtcttttagcttactacgaagtttgtacatttttaaatgaaggagctacagaagtttgggatgcaccacaagaagtaccttatgcataccagggtaatgaatgggttggatacgataatgtacgtagttttaagcttaaggctcaatggcttaaggataataatttaggtggagcagttgtatggccacttgatatggatgatttttctggtagtttttgtcatcaacggcactttcctcttacttcaacacttaagggtgatcttaatattcattcagcatcttgtaagggaccatattaactcgag

SEQ ID NO: 14 - protein, 405 aa, Mus musculus
MAKFWKKALLTIAALTVGTSAGITSVSAYQLMCYYTSWAKDRPIEGSFKPGNIDPCLCTHLIYAFAGMQNNEITYTHEQDLRDYEALNGLKDKNTELKTLLAIGGWKFGPAPFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 15 - protein, 398 aa, Mus musculus
MAKLILVTGLAILLNVQLGSSYQLMCYYTSWAKDRPIEGSFKPGNIDPCLCTHLIYAFAGMQNNEITYTHEQDLRDYEALNGLKDKNTELKTLLAIGGWKFGPAPFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 16 - DNA, 1181 bp, Homo sapiens
  source 1..1181: mol_type = unassigned DNA
  CDS 3..1175: protein_id = 33; translation = MAKFWKKALLTIAALTVGTSAGITSVSAYKLVCYYTSWSQYREG DGSCFPDALDRFLCTHIIYSFANISNDHIDTWEWNDVTLYGMLNTLKNRNPNLKTLLS VGGWNFGSQRFSKIASNTQSRRTFIKSVPPFLRTHGFDGLDLAWLYPGRRDKQHFTTL IKEMKAEFIKEAQPGKKQLLLSAALSAGKVTIDSSYDIAKISQHLDFISIMTYDFHGA WRGTTGHHSPLFRGQEDASPDRFSNTDYAVGYMLRLGAPASKLVMGIPTFGRSFTLAS SETGVGAPISGPGIPGRFTKEAGTLAYYEICDFLRGATVHRTLGQQVPYATKGNQWVG YDDQESVKSKVQYLKDRQLAGAMVWALDLDDFQGSFCGQDLRFPLTNAIKDALAAT
ccatggcaaaattttggaagaaagcactattaacaattgcagccttaacagtcggcacctccgcaggaattacaagcgtttctgcctacaagcttgtttgttactacacttcatggtctcaataccgagaaggtgatggaagttgttttccagatgctcttgatcggtttttatgtacacatattatttactcttttgcaaatattagtaatgatcacattgatacatgggaatggaatgatgttactctttacggtatgcttaatacacttaagaatcgtaatccaaatttaaagactttacttagtgtaggtggatggaattttggttctcaacggtttagtaagattgcttcaaatactcaatctcgtcggacatttattaaaagtgttccaccatttttacgtactcatggttttgatggacttgatttagcatggctttatccaggtcgacgtgataagcaacactttactacacttattaaagaaatgaaggctgaatttattaaggaagcacaacctggtaaaaagcaacttcttcttagtgctgcattatcagctggaaaggttactattgatagttcatacgatattgcaaagattagtcaacatcttgattttatttcaattatgacatacgattttcacggtgcttggcggggtactacaggacatcacagtccattatttcgtggacaagaagatgcttcacctgatcggttttctaatactgattatgcagttggttacatgcttcggttaggagctccagcatctaaacttgtaatgggtattcctacttttggacgatcatttacattagcttctagtgaaactggtgttggagcaccaatttcaggtccaggaattcctggtcgttttactaaggaagctggaacacttgcatactacgaaatttgtgattttcttcggggtgctactgttcatcgaacacttggacaacaagtaccttatgcaactaaaggtaatcaatgggttggatacgatgatcaagaaagtgttaagtcaaaggtacaataccttaaggatcgacaattagctggtgcaatggtatgggctcttgatcttgatgattttcaaggtagtttttgtggacaagatcttcgttttccacttactaatgctattaaggatgcattagctgcaacataactcgag

SEQ ID NO: 17 - protein, 390 aa, Homo sapiens
MAKFWKKALLTIAALTVGTSAGITSVSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRTLGQQVPYATKGNQWVGYDDQESVKSKVQYLKDRQLAGAMVWALDLDDFQGSFCGQDLRFPLTNAIKDALAAT

SEQ ID NO: 18 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRTLGQQVPYATKGNQWVGYDDQESVKSKVQYLKDRQLAGAMVWALDLDDFQGSFCGQDLRFPLTNAIKDALAAT

SEQ ID NO: 19 - protein, 20 aa, Lactobacillus sp.
MAGNSSNFIHKIIKQIFTHR

SEQ ID NO: 20 - DNA, 7673 bp, synthetic construct
  misc_feature 1..7673: note = Recombinant plasmid
  source 1..7673: mol_type = other DNA
  CDS 5853..7658: protein_id = 34; translation = MVRPVETPTREIKKLDGLWAFSLDRENCGIDQRWWESALQESRA IAVPGSFNDQFADADIRNYAGNVWYQREVFIPKGWAGQRIVLRFDAVTHYGKVWVNNQ EVMEHQGGYTPFEADVTPYVIAGKSVRITVCVNNELNWQTIPPGMVITDENGKKKQSY FHDFFNYAGIHRSVMLYTTPNTWVDDITVVTHVAQDCNHASVDWQVVANGDVSVELRD ADQQVVATGQGTSGTLQVVNPHLWQPGEGYLYELCVTAKSQTECDIYPLRVGIRSVAV KGEQFLINHKPFYFTGFGRHEDADLRGKGFDNVLMVHDHALMDWIGANSYRTSHYPYA EEMLDWADEHGIVVIDETAAVGFNLSLGIGFEAGNKPKELYSEEAVNGETQQAHLQAI KELIARDKNHPSVVMWSIANEPDTRPQGAREYFAPLAEATRKLDPTRPITCVNVMFCD AHTDTISDLFDVLCLNRYYGWYVQSGDLETAEKVLEKELLAWQEKLHQPIIITEYGVD TLAGLHSMYTDMWSEEYQCAWLDMYHRVFDRVSAVVGEQVWNFADFATSQGILRVGGN KKGIFTRDRKPKSAAFLLQKRWTGMNFGEKPQGGKQ
gaattcggtaccccgggttcgaaggcgccaagcttcaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaagcgagaataaggaagataaatcccataagggcgggagcagaatgtccgagactaattcatgagatcgattttttattaaaacgtctcaaaatcgtttctgagacgttttagcgtttatttcgtttagttatcggcataatcgttaaaacaggcgttatcgtagcgtaaaagcccttgagcgtagcgtgctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaataaaaaaaataagcgaaagctcgcgtttttagaaggatacgagttttcgctacttgtttttgataaggtaatatatcatggctattaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgaagggggcttttattttggtttgatgttgcgattaatagcaatacgattgcaataaacaaaatgatccccttagaagcaaacttaagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaatatctcaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggggtcgacccttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttattaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtagcgcctgcaggcacgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtttaatttgaaaattgatattagcgtttaacagttaaattaatacgttaataatttttttgtctttaaatagggatttgaagcataatggtgttatagcgtacttagctggccagcatatatgtattctataaaatactattacaaggagattttagccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagggaggcaaacaatgatctagactcgag

SEQ ID NO: 21 - protein, 602 aa, synthetic construct
  REGION 1..602: note = Synthetic Construct
MVRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQGGKQ

SEQ ID NO: 22 - DNA, 22 bp, synthetic construct
  misc_feature 1..22: note = PCR primer
  source 1..22: mol_type = other DNA
gcagccttaacagtcggcacct

SEQ ID NO: 23 - DNA, 23 bp, synthetic construct
  misc_feature 1..23: note = PCR primer
  source 1..23: mol_type = other DNA
acgtgcaacaatctgcaaagcac

SEQ ID NO: 24 - DNA, 2999 bp, synthetic construct
  misc_feature 1..2999: note = pVAX vector
  source 1..2999: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 25 - DNA, 347 bp, synthetic construct
  misc_feature 1..347: note = insert
  source 1..347: mol_type = other DNA
gctagcatggacgccaaggtcgtcgccgtgctggccctggtgctggccgcgctctgcatcagtgacggtaaaccagtcagcctgagctaccgatgcccctgccggttcttcgagagccacatcgccagagccaacgtcaagcatctgaaaatcctcaacactccaaactgtgcccttcagattgttgcacggctgaagaacaacaacagacaagtgtgcattgacccgaaattaaagtggatccaagagtacctggagaaagccttaaacaagggatcaggtgccacgaacttctctctgttaaagcaagcaggagacgtggaagaaaaccccggtcccaaaagctt

SEQ ID NO: 26 - DNA, 5395 bp, synthetic construct
  misc_feature 1..5395: note = vector
  source 1..5395: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttcgccaccatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctgcgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctaccacagcttcgtggtggacaaccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgcgagggcagaggaagtcttctaacatgcggtgacgtggaggagaatcccggccctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 27 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 28 - protein, 96 aa, Mus musculus; identical to SEQ ID NO: 2

SEQ ID NO: 29 - protein, 96 aa, Homo sapiens; identical to SEQ ID NO: 5

SEQ ID NO: 30 - protein, 125 aa, Mus musculus; identical to SEQ ID NO: 8

SEQ ID NO: 31 - protein, 125 aa, Homo sapiens; identical to SEQ ID NO: 11

SEQ ID NO: 32 - protein, 405 aa, Mus musculus; identical to SEQ ID NO: 14

SEQ ID NO: 33 - protein, 390 aa, Homo sapiens; identical to SEQ ID NO: 17

SEQ ID NO: 34 - protein, 602 aa, synthetic construct; identical to SEQ ID NO: 21